(19) United States
(12) Patent Application Publication
(10) Pub. No.: US 2015/0148784 A1
(43) Pub. Date: May 28, 2015

(54) VACUUM-ASSISTED DRAINAGE SYSTEM

(71) Applicants: Steffen Suess, Halberstadt (DE); Harry Leibitzki, Blankenburg (DE)

(72) Inventors: Steffen Suess, Halberstadt (DE); Harry Leibitzki, Blankenburg (DE)

(21) Appl. No.: 14/363,237

(22) PCT Filed: Jan. 24, 2012
(86) PCT No.: PCT/DE2012/000088

§ 371 Date: Feb. 5, 2015

(30) Foreign Application Priority Data
Dec. 9, 2011 (DE) 10 2011 121 072.9

(51) Int. Cl. A61F 13/00; A61M 1/00; A61F 13/02

(52) U.S. Cl. CPC A61F 13/00068; A61F 13/0216; A61M 1/0088; A61F 2013/00174; A61F 2013/00412; A61F 2013/00536
USPC 604/543

(57) ABSTRACT

A vacuum-assisted drainage system includes a first porous pad that is permeable to liquids and has a through opening in which a fistula adapter is placed on a film provided with perforations, a cover film for covering the first pad and the wound, and for providing a substantially airtight seal around the wound, wherein in the cover film an opening is provided that surrounds the fistula adapter in a substantially airtight manner, and a second porous pad that is permeable to liquids is positioned partially above the first pad on the cover film and is provided with a sealing film for covering the second pad and for providing a substantially airtight connection to the cover film and around the wound, wherein between the first pad and the second pad air guiding routes are provided that pass through the cover film, and an open drainage line is located within the second pad.

### BACKGROUND OF THE INVENTION

The invention relates to a vacuum-assisted drainage system,

In wound drainage systems, a distinction is made between direct drainages (flexible drainage tube is lying directly in the wound) and sponge-mediated drainages (flexible drainage tube is placed in a sponge that is lying in the wound).

The direct wound drainage has been known for decades. Systems for wound drainages with sponges that are introduced into the wound are based on the state of the art and were developed more than 60 years ago.

DE 847 475 discloses, for example, a system for draining wounds by using soft tampons. In such a system, one end of a suction line (or several of such suction lines) is (are) embedded in a tampon and is (are) open therein, and said tampon is preferentially made of fine-pored and soft-elastic natural, rubber, foam rubber or cellulose sponge or of gauze, cotton wool or the like. In such a system, the suction line can be subdivided via a fork-shaped piece in several branches leading into the tampon. Additionally, this system can be equipped with further tubes (irrigation lines) for the supply of irrigation, disinfection or medical liquids to the wound.

DE 696 13 997 T2 describes a portable wound treating system comprising a porous, liquid-permeable pad to be introduced into the wound, a dressing for covering the wound and ensuring a substantially air-tight seal around the wound, a drainage tube connecting the pad with a suction pump, and at least, one filter so that liquids can be sucked from the wound. Moreover, a pressure control device is provided to monitor the pressure mainly at the wound area.

However, all known sponge-mediated drainage systems have the disadvantage that the liquid-permeable pad (the sponge), after being introduced into the wound, can be clogged by solid substances which may be present in the liquids and settle in the pad. As a result, the pad completely fails to fulfill its drainage function.

This problem is solved in DE 10 2004 053 166 A1 in which a vacuum-assisted drainage system is described that consists of a porous, liquid-permeable pad to be introduced into a wound, a dressing for covering the pad and the wound and for providing a substantially air-tight seal around the wound, and a drainage line connecting the pad with a suction pump so that liquids can be sucked from the wound. The pad is provided with a through opening in which a rigid, tube-shaped spacer is positioned that can be introduced into the opening and a second drainage line can be fed through the opening of the spacer.

The disadvantage of this technical solution is that for smaller wounds only an insufficient access is provided to the pad for the suction process in fistulotomy procedures.

From US 2001/0043943 A1 a drainage solution is known in which the direct drainage is combined with a sponge-mediated drainage, too.

This solution has also the disadvantage that for smaller wounds only an insufficient access is provided to the pad for the suction process in fistulotomy procedures.

### SUMMARY OF THE INVENTION

The object of the invention is to make available a vacuum-assisted drainage system that prevents the clogging of the liquid-permeable pad (of the sponge) during fistulotomy procedures for smaller wounds, ensures a drainage effect even if the liquids to be discharged have solid components and only an insufficient access is provided to the pad for the suction process.

According to the invention, this object is achieved by the characteristic features of the first claim and supplemented by advantageous embodiments according to the subclaims.

The essence of the invention consists in that the vacuum-assisted drainage system comprises a first porous pad that is permeable to liquids and has a through opening in which a fistula adapter is placed, said pad and fistula adapter are carried by a perforated film to be inserted into a wound, said film air-tightly communicates with a. cover film used to cover the pad and the wound and to ensure a substantially air-tight sealing around the wound, said cover film has an opening that surrounds the fistula adapter in a substantially air-tight manner, and a second porous pad that is permeable to liquids is positioned partially above the first pad on the cover film and is provided with a sealing film for covering the pad and providing a substantially air-tight sealing to the cover film and around the wound.

An essential feature of the invention is that air guiding routes passing through the cover film are provided between the first pad and the second pad.

Within the second pad an open drainage line is arranged and can be connected with a suction pump so that liquids can be removed from the wound by suction.

Alternatively, a drainage port with air guiding routes passing through the cover film can be positioned above the pad and ends in a drainage line, which can be connected with a suction pump so that liquids can be removed from the wound by suction.

In this technical solution, the first pad and the second pad, which is arranged opposite to the first pad in a space-shifted manner (and is considered a kind of bridge to the drainage line), form a liquid-guiding line in fistulotomy for smaller wounds, and said line leads beyond the wound area so that the for suction purposes otherwise only insufficient access to the first pad has been overcome.

Via the fistula adapter a drainage bag can be coupled or a further drainage line can be inserted into the wound to remove more solid substances from the wound.

To register a possible vascular injury during the use of the vacuum-assisted drainage system, for example at night, this drainage system can he provided with a blood detector, e.g. a spectroscope with optical fiber the detection end of which ends in the drainage line.

### BRIEF DESCRIPTION OF THE DRAWINGS

In the following, the invention is explained in more detail by means of the schematic drawings. They show:

FIG. 1 a first embodiment of the inventive vacuum-assisted drainage system in a schematic sectional representation of the lateral view,

FIG. 2 a second embodiment of the inventive vacuum-assisted drainage system in a schematic sectional representation of the lateral view, and

FIG. 3 the embodiment according to FIG. 2 in a schematic top view.

### DETAILED DESCRIPTION OF THE INVENTION

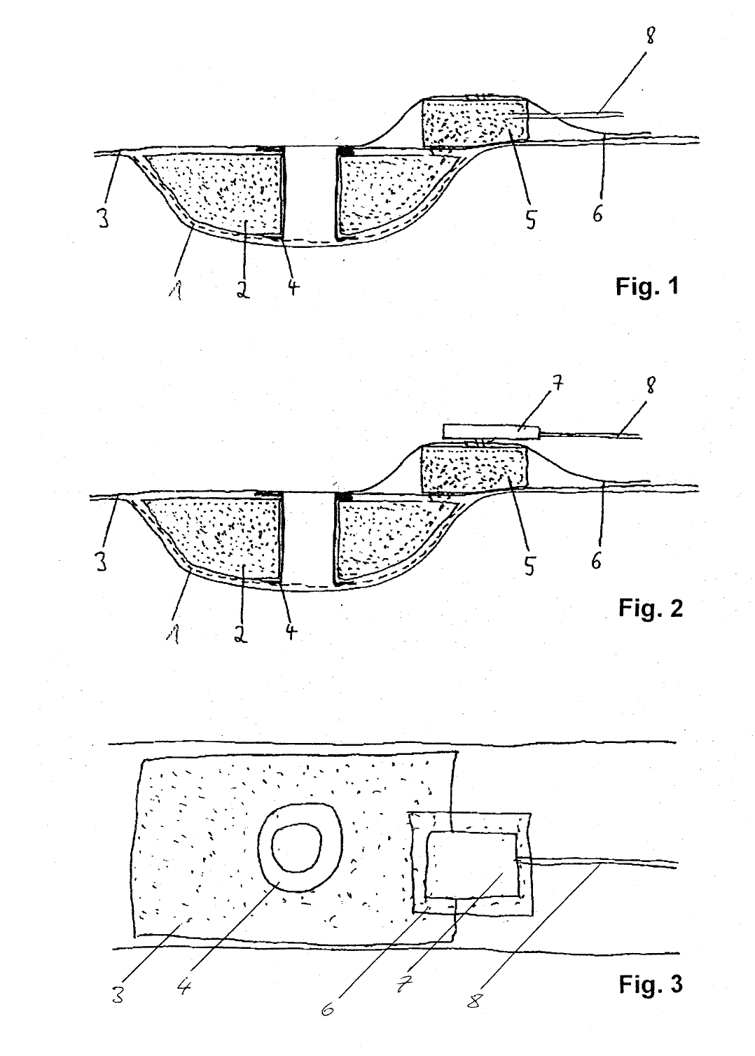

The vacuum-assisted drainage system shown in FIG. 1 consists of a first porous pad (2) permeable to liquids and a second porous pad (5) permeable to liquids.

The pad (2) is provided with a through opening in which a fistula adapter (4) is placed and a film (1) provided with perforations carries the pad (2) and the fistula adapter (4) to insert them into a wound.

Said film (1) is air-tightly connected with a cover film (3) used to cover the pad (2) and the wound and to ensure a substantially air-tight sealing around the wound, and the cover film (3) has an opening that surrounds the fistula adapter (4) in a substantially air-tight manner.

The second pad (5) is positioned partially above the first pad (2) on the cover film (3) and is provided with a sealing film (6) for covering the pad (5) and providing a substantially air-tight connection to the cover film (3) and around the wound.

An essential feature of the invention is that air guiding routes passing through the cover film (3) are provided between the first pad (2) and the second pad (5).

This design allows to arrange a first drainage line via the first pad (2) and the second pad (5) for draining highly fluent and fluent body secretions, such as lymph, blood or urine, out of the wound.

An open drainage line (8) is arranged within the pad (5) and can be connected with a suction pump so that liquids can be removed from the wound by suction,

Alternatively, as shown in FIG. 2, the open drainage line (8) arranged within the pad (5) can be replaced by a drainage port (7) that is positioned above the pad (5) and provided with air guiding routes passing through the cover film (6), and the drainage port (7) can end in a drainage line (8) which can be connected with a suction pump so that liquids can be removed by suction from the wound.

Due to the drainage port (7) the cross section of the drainage line (8) lying against the pad (5) is enlarged.

The fistula adapter (4) allows to place a second drainage line to drain solid/liquid body secretions, such as excrements or pus, out of the wound with this part of the area being substantially air-tightly separated from the area to be sucked.

A drainage bag not shown in the figures can be coupled to the fistula adapter (4).

If necessary, a second drainage line not shown in the figures can be led through the opening of the fistula adapter (4).

Preferentially, this fistula adapter (4) is made of plastic material and its inner diameter ranges from 1 to 5 cm and can be freely selected depending on the application.

It is particularly advantageous if the pads (2) and (5) consist of solidified plastic foam with a pore size ranging from 0.3 to 2.5 m.

This foam can be, for example, polyurethane foam with a pore size of between 0.35 to 2.3 mm or polyvinyl alcohol foam with a pore size of between 0.4 and 2 mm.

The cover film (3) and the sealing film (6) are preferentially provided with an adhesive layer (not shown in the figures) so that a good air-tight sealing is ensured.

Advantageously, the suction pump (not shown in the figures) generates a negative pressure of between 50 mm Hg and 375 mm Hg provided there is air-tight sealing of the inventive system around the wound.

To register a possible vascular injury during the use of the vacuum-assisted drainage system, for example at night, the drainage system, can be provided with a blood detector, e.g. a spectroscope with optical fiber the detection end of which ends in the drainage line (8).

This invention avoids the clogging of the liquid-permeable pads (2) and (5) by solid substances such as excrements by discharging them through the fistula adapter (4) so that the effect of the drainage system will also be ensured, if the liquids to be removed include solids content.

The optionally provided blood detector (not shown in the figures) makes it possible to register vascular injuries caused by the employment of the drainage system during the suction process.

The inventive vacuum-assisted drainage system is used, for example, for fistulotomy.

For this purpose, the fistula adapter (4) is placed above the fistula (with this area being largely air-tightly separated from the area to be sucked) and the liquid ichor around the fistula adapter (4) is sucked via the pads (2) and (5) (which have a substantially air-tight connection to the wound surrounding due to the cover film (3) and the sealing film (6)) through the drainage line (8), and more solid fistula secretion can be directly discharged or guided into a drainage bag (not shown in the figures) because the fistula adapter (4) is placed directly close to the fistula.

Depending on the situation of the wound it is recommended to replace the inventive vacuum-assisted drainage system by a new one every two to five days when changing the dressing,

The use of the inventive vacuum-assisted drainage system avoids the clogging of the liquid-permeable pad (the sponge) during fistulotomy procedures for smaller wounds and guarantees a drainage effect even if die liquids to be discharged include solids content and only an insufficient access is provided to the pad for the suction process.

The inventive vacuum-assisted drainage system has the advantage of high user-friendliness and patient comfort.

The individual features of the invention or any combination of them indicated in the description, the following claims and the drawings can be decisive for the invention.

1. A vacuum-assisted drainage system comprising
a first porous pad that is permeable to liquids and has a through opening in which a fistula adapter is placed, a perforated film carrying the pad and the fistula adapter to be inserted into a wound, a cover film with which said perforated film is air-tightly connected and which covers the pad and the wound and ensures a substantially air-tight sealing around the wound, the cover film haying an opening that surrounds the fistula adapter (4) in a substantially air-tight manner, and
a second porous pad that is permeable to liquids and is positioned on the cover film with a part of the second pad directly above the first pad, a sealing film covering the second pad and providing a substantially air-tight connection to the cover film and around the wound, wherein
air guiding routes are provided between the first pad and the second pad and pass through the cover film, and either
an open drainage line is lying within the pad and can be connected with a suction pump so that liquids can be removed from the wound by suction, or
positioned above the second pad is a drainage port with air guiding routes passing through the sealing film said drainage port communicating with an open drainage line that can be connected with a suction pump so that liquids can be removed from the wound by suction.

2. (canceled)

3. The vacuum assisted drainage system according to claim 1, wherein the fistula adapter has an internal diameter of between 1 and 5 cm.

4. The vacuum assisted drainage system according to claim 3, wherein the fistula adapter is made of plastic material.

5. The vacuum-assisted drainage system according to claim 1, wherein the first and second pads are made of solidified plastic foam with a pore size ranging from 0.3 to 2.5 mm.

6. The vacuum-assisted drainage system according to claim 5, wherein the foam is polyurethane foam with a pore size of between 0.35 and 2.3 mm.

7. The vacuum-assisted drainage system according to claim 5, wherein the foam is polyvinyl alcohol foam with a pore size of between 0.4 and 2 mm.

8. The vacuum-assisted drainage system according to claim 1, wherein the cover film and the sealing film are provided with an adhesive layer.

9. (canceled)

10. The vacuum-assisted drainage system according to claim 11, the negative pressure applied by the pump is between 50 mm Hg and 375 mm Hg when the drainage system is air-tightly sealed.

11. A method of treating a wound, comprising applying the drainage system of claim 1 to the wound, connecting a suction pump to the open drainage line and operating the pump to apply a negative pressure to the drainage line.
